(19) United States
(12) Patent Application Publication
(10) Pub. No.: US 2024/0188854 A1
(43) Pub. Date: Jun. 13, 2024

(54) METHOD OF DETERMINING A CURRENT GLUCOSE VALUE IN A TRANSPORT FLUID

(71) Applicant: EYESENSE GMBH, Großostheim (DE)

(72) Inventors: Theresa KRUSE, Ulm (DE); Knut GRAICHEN, Blaubeuren (DE); Roland KRIVANEK, Großostheim (DE); Achim MUELLER, Großostheim (DE)

(73) Assignee: EYESENSE GMBH, Großostheim (DE)

(21) Appl. No.: 18/283,371

(22) PCT Filed: Mar. 22, 2022
(86) PCT No.: PCT/DE2022/200052

§ 371 Date: Sep. 21, 2023

(30) Foreign Application Priority Data
Mar. 22, 2021 (DE) 10 2021 202 767.9

(51) Int. Cl. A61B 5/145; G16H 50/30; G16H 50/50

(52) U.S. Cl. CPC A61B 5/14532; G16H 50/30; G16H 50/50

(57) ABSTRACT

The invention relates to a method for, in particular, continuously determining a current glucose value in a transport fluid, in particular blood, of an organism, comprising the steps of
a) Determining a measurement series using a sensor device, comprising at least two measured values for a tissue glucose value that are spaced apart in time in the tissue surrounding the transport fluid,
b) determining the tissue glucose value using the determined series of measurements based on a measurement model in the form of a linear or non-linear function, with the measurement model measuring values of the sensor device measuring tissue glucose values taking into account at least one measurement noise value are assigned,
c) providing at least one state transition model, with the at least one state transition model being used to assign at least one glucose value in the transport fluid to the determined tissue glucose values, taking into account at least one process noise value, and
d) estimating the current glucose value in the transport fluid based on an approximation of at least one provided state transition model and the determined tissue glucose value using at least one Kalman filter in the case of a measurement model in the form of a linear function or at least one extended Kalman filter Case of a measurement model in the form of a non-linear function.

The invention relates to a method for determining, in particular continuously, a current glucose value in a transport fluid, in particular blood, of an organism.

The invention further relates to a device for, in particular, continuously determining a current glucose value in a transport fluid, in particular blood, of an organism.

The invention also relates to an evaluation device for determining, in particular continuously, a current glucose value in a transport fluid, in particular blood, of an organism.

The invention further relates to a non-volatile, computer-readable medium for storing instructions which, when executed on a computer, cause a method for, in particular, continuous determination of a current glucose value in a transport fluid, in particular blood, of an organism to be carried out.

Although the present invention is generally applicable to any method for determining a current glucose value in a transport fluid, the present invention will be explained in relation to the blood glucose concentration in an organism.

Systems for continuous glucose monitoring, also known as CGM—Continuous Glucose Monitoring—have become known for determining a blood glucose concentration BG in an organism, particularly in humans. In a CGM system, an interstitial tissue glucose concentration IG is typically measured automatically, for example every one to five minutes. In particular, diabetic patients benefit from CGM systems because compared to self-monitoring procedures—also known as self-monitoring procedures SMBG—in which the patient manually determines the blood glucose value four to ten times a day, measurements can be carried out with a significantly higher frequency. This enables automated evaluations and warning signals to be sent to the patient, especially while the patient is sleeping, which helps to avoid critical health conditions in patients.

Known CGM systems are based on the one hand on electrochemical processes. Such a CGM system is described, for example, in WO 2006/017358 A1. In addition, optical CGM systems have become known, for example from DE 10 2015 101 847 B4, in which a fluorescence dependent on the glucose value is used and which is hereby incorporated by reference. Both types of CGM systems measure an interstitial tissue glucose concentration.

It is also known that the tissue glucose concentration or interstitial glucose concentration IG differs from the blood glucose concentration, hereinafter abbreviated as BG. A large deviation exists in particular after strong influences on the blood glucose level, for example through food or nutrient intake or when administering insulin, as described in the non-patent literature Basu, Ananda et al. “Time lag of glucose from intravascular to interstitial compartment in humans” (Diabetes (2013): DB-131132). This deviation is caused by a diffusion process in the tissue surrounding the blood, so that the IG value follows the BG value with a time delay and is dampened, which is described, for example, in the non-patent literature Rebrin, Kerstin et al. “Subcutaneous glucose predicts plasma glucose independent of insulin: implications for continuous monitoring” (American Journal of Physiology-Endocrinology and Metabolism 277.3 (1999): E561-E571).

Due to the damping and time delay described between the two glucose concentrations, on the one hand in the blood BG and on the other hand in the surrounding tissue IG, a calibration of the CGM system using a manual determination of the blood glucose concentration, for example by a drop of blood extracted from a finger and the glucose concentration in the drop of blood is determined using an extemal measuring device, leads to significant inaccuracies.

In order to achieve an accurate calibration of the CGM system, however, the previously described difference between tissue glucose concentration and blood glucose concentration must be taken into account or at least estimated. Various methods have become known for this. From the non-patent literature Keenan, D. Barry et al. “Delays in minimally invasive continuous glucose monitoring devices: a review of current technology.” (Journal of diabetes Science and technology 3.5 (2009): 1207-1214) it has become known to use a time-delayed glucose signal for calibration. Furthermore, it is known from the non-patent literature Knobbe, Edward J. and Bruce Buckingham “The extended Kalman filter for continuous glucose monitoring.” (Diabetes technology & therapeutics 7.1 (2005): 15-27) to compensate for the damping and time delay of the diffusion process of glucose between blood and tissue by means of a Kalman filter.

The problem here, however, is that mobile devices for the continuous determination of current glucose values have limited computing and energy resources. For the determination of the current glucose values, only a comparatively small amount of computing power is available, coupled with limited energy, so that the previously known methods cannot be carried out on mobile devices, or can only be carried out for a short time, which considerably limits their usefulness.

It is therefore an object of the present invention to specify a method, a device and an evaluation device, which enable a more precise determination of the glucose value, in particular in blood, with fewer resources and simpler implementation.

A further object of the present invention is to specify an alternative method, an alternative device and an alternative evaluation device.

A further object of the present invention is to provide a method, a device and an evaluation device with an improved determination of the blood glucose concentration in an organism based on a measurement of interstitial tissue glucose level.

In one embodiment, the present invention solves the above-mentioned objects by a method for, in particular, continuously determining a current glucose value in a transport fluid, in particular blood, of an organism, comprising the steps
a) Determining a measurement series using a sensor device, comprising at least two measured values for a tissue glucose value in the tissue surrounding the transport fluid,
b) determining the tissue glucose value using the determined measurement series based on a measurement model in the form of a linear or non-linear function, with the measurement model being used to assign measured values of the sensor device to tissue glucose values, taking into account at least one measurement noise value,
c) providing at least one state transition model, with the at least one state transition model being used to assign at least one glucose value in the transport fluid to the determined tissue glucose values, taking into account at least one process noise value, and
d) estimating the current glucose value in the transport fluid based on an approximation of at least one provided state transition model and the determined tissue glucose value using at least one Kalman filter in the case of a measurement model in the form of a linear function or at least one extended Kalman filter in the case of a measurement model in the form of a non-linear function.

In a further embodiment, the present invention achieves the above-mentioned objects by means of a device for, in particular, continuously determining a current glucose value in a transport fluid, in particular blood, of an organism, preferably suitable for carrying out a method according to one of claims 1-15, comprising a sensor device, in particular for measuring fluorescence in tissue surrounding the transport fluid by means of a probe, in particular a polymer-optical fiber probe, designed to determine a series of measurements,
at least two measured values spaced apart in time for a tissue glucose value in the tissue surrounding the transport fluid,
a provision device designed to provide at least one state transition model, with the at least one state transition model being used to assign at least one glucose value in the transport fluid to the determined tissue glucose values, taking into account at least one process noise value, and to provide a measurement model in the form of a linear or non-linear function, with the measurement model providing measured values from the sensor device tissue glucose values are assigned taking into account at least one measurement noise value, and
an evaluation device, designed to determine the tissue glucose value using the determined series of measurements based on the measurement model and to estimate the current glucose value in the transport fluid based on an approximation of at least one provided state transition model and the determined tissue glucose value using at least one Kalman filter in the case of a measurement model in the form of a linear function or at least one extended Kalman filter in the case of a measurement model in the form of a non-linear function.

In a further embodiment, the present invention achieves the above-mentioned objects with an evaluation device for, in particular, continuously determining a current glucose value in a transport fluid, in particular blood, of an organism, comprising
at least one interface for connecting a sensor device for providing a series of measurements, comprising at least two measured values for a tissue glucose value in the tissue surrounding the transport fluid,
at least one memory for storing at least one state transition model, wherein the at least one state transition model is used to assign the tissue glucose values determined by the at least one state transition model to at least one glucose value in the transport fluid to, taking into account at least one process noise value, and for storing a measurement model in the form of a linear or non-linear function, with the measurement model being used to assign measured values of the sensor device to tissue glucose values, taking into account at least one measurement noise value, and
a computing device designed to determine the tissue glucose value using the determined series of measurements based on the stored measurement model and to estimate the current glucose value in the transport fluid based on an approximation of at least one provided state transition model and the determined tissue glucose value using at least one Kalman filter in the case of a measurement model in the form of a linear function or at least one extended Kalman filter in the case of a measurement model in the form of a non-linear function.

In a further embodiment, the present invention achieves the above-mentioned objects by a non-transitory, computer-readable medium for storing instructions which, executed on a computer, cause a method for, in particular, continuous determination of a current glucose value in a transport fluid, in particular blood, of an organism, to be carried out, preferably suitable for carrying out a method according to any one of claims 1-15, comprising the steps
a) Determining, by means of a sensor device, a series of measurements comprising at least two time-spaced ones measured values for a tissue glucose value in the tissue surrounding the transport fluid,
b) determining the tissue glucose value using the determined series of measurements based on a measurement model in the form of a linear or non-linear function, with the measurement model being used to assign measured values of the sensor device to tissue glucose values, taking into account at least one measurement noise value,
c) providing at least one state transition model, with the at least one state transition model using the determined tissue glucose at least one glucose value in the transport fluid is assigned to glucose values, taking into account at least one process noise value, and
d) estimating the current glucose value in the transport fluid based on an approximation of at least one provided state transition model and the determined tissue glucose value using at least one Kalman filter in the case of a measurement model in the form of a linear function or at least one extended Kalman filter in the case of a measurement model in the form of a nonlinear function.

In other words, a method for determining a blood glucose concentration in an organism is proposed. This has the following procedural steps:

In a first method step, a measurement series is recorded with at least two sensor measurement values spaced apart in time of an interstitial tissue glucose value of the tissue of the organism by means of one or more sensors.

In a further step, a measurement or sensor model of the relation between the sensor measurement values and the tissue glucose value is provided and one or more state transition models are provided which include models for the relation between the tissue glucose value and the blood glucose value.

In a further method step, the blood glucose value of the organism is quantified by means of an estimate based on an approximation of the state transition model and the tissue glucose values, wherein it is essential that the estimation is carried out using at least one Kalman filter in the case of a measurement model in the form of a linear function or using at least one extended Kalman filter in the case of a measurement model in the form of a non-linear function.

The Kalman filter is an unbiased and consistent estimator with minimal variance. Because of these estimation properties, the Kalman filter is an optimal linear filter. In contrast to other (recursive) linear estimators, which also minimize least squares, the Kalman filter also allows the treatment of problems with correlated noise components.

The extended Kalman filter is a non-linear extension of the Kalman filter described above. The extended Kalman filter approximates the nonlinear problem analytically based on the nonlinear function by a linear problem.

In this case, the evaluation device can in particular be a computer, an integrated circuit or the like, which is designed in particular for optimized calculation, for example the trace of a matrix. The device and/or evaluation device can be designed as a portable device with an independent energy source, for example a battery, a rechargeable battery or the like, which keeps efficient operation, and therefore the energy consumption for carrying out the method according to an embodiment of the present invention, as low as possible in order to enable battery operation for as long as possible, which improves the user experience. In particular, power-saving processors, circuits, switching circuits, interfaces, in particular wireless interfaces or the like can be used for this purpose. The implementation of the method can be adapted in particular with regard to its parameters, for example to the underlying device or evaluation device, for example with regard to the evaluation horizon and/or the noise horizon, the scope of random samples, the linear or non-linear functions or the like, which is described below in order to achieve sufficient accuracy on the one hand and a long running time on the other hand.

One of the possible advantages that can be achieved with the embodiments is that it enables the current glucose value in the transport fluid, in particular blood, to be estimated in a way that is efficient in terms of time and computer resources. Another advantage is that the flexibility is significantly increased compared to known methods, since there are no restrictions on specific sensor models and/or state transition models. A further advantage is that not only is the accuracy of the current glucose value increased, but past glucose values are also improved at the same time.

Other features, advantages, and other embodiments of the invention are described below or will become apparent thereby.

According to a preferred embodiment of the invention, a plurality of state transition models are provided which, depending on the course over time of the estimated current glucose value, in particular its rate of change over time, change. This enables in particular an efficient and at the same time precise determination of the current glucose value.

According to a further preferred embodiment of the invention, at least two state transition models are provided, one based on a constant glucose concentration, one based on a constant change in glucose concentration and/or one based on a weighted sum of previous glucose concentrations. This enables the current glucose value to be determined in a particularly resource-efficient manner, since the state transition model can be adapted based on the dynamics of the glucose value. This avoids overestimating or underestimating the glucose value in the blood when the glucose value rises or falls.

According to a further preferred embodiment of the invention, determined values are filtered by means of at least one filter function, wherein errors, in particular measurement errors, of the sensor device being suppressed by means of the at least one filter function. Using the filter function, faulty measurements, for example sensor errors or outliners in the measured values, can be sorted out in a simple manner, i.e. they are not taken into account in the further calculation of the current glucose value.

According to a further preferred embodiment of the invention, the at least one measurement noise value is adjusted, in particular regularly. This ensures that the respective noise values are adjusted efficiently, particularly at regular intervals, in order on the one hand to ensure that the current glucose value is sufficiently accurate and on the other hand to avoid unnecessary adjustments or updates, which are not or only marginally reflected in an increase in the accuracy of the current glucose value.

According to a further preferred embodiment of the invention, in order to adapt the at least one measurement noise value, the variance of the measurement noise is determined using a random sample of measured values, in particular the variance is estimated. A possible advantage of this is that the measurement noise value can be adjusted in an efficient way.

According to a further preferred embodiment of the invention, a statistical test, in particular a Kolmogorov-Smimov-test, is used to check whether the null hypothesis—the random sample follows a mean-free Gaussian distribution with the determined variance of the measurement noise—is not rejected. One of the possible advantages is that it enables the measurement noise to be adapted in a simple manner.

According to a further preferred embodiment of the invention, the variance of the measurement noise is determined for at least one further sample of measured values as long as the null hypothesis is rejected. A possible advantage is that it is an efficient way of adjusting the measurement noise.

According to a further preferred embodiment of the invention, the at least one filter function is used to check measured values for outliners and measured values that were determined as outliners are discarded, in particular using an NIS test. A possible advantage is that the accuracy of the determination of the current glucose value is further improved.

According to a further preferred embodiment of the invention, the at least one filter function is used to check the measured values for exceeding and/or falling below specified limit values before they are discarded. This represents a particularly simple way of checking measured values for outliners.

According to a further preferred embodiment of the invention, a current measured value that was not determined as an outliner is nevertheless rejected as a measurement error if at least a predefined number, in particular two chronologically consecutive, earlier measured values, were rejected as measurement errors. This means that a fault in the measuring system is assumed, which is only considered to have ended when all conditions or limit values are met. A possible benefit of this is that it further improves the accuracy of determining the current glucose value.

According to a further preferred embodiment of the invention, the state transition model includes a diffusion model for time-dependent modelling of the diffusion process of glucose from the transport fluid into the surrounding tissue. Using a diffusion model, in particular based on a diffusion constant, it is possible to model the damping and time delay between the glucose value in the transport fluid, in particular in the blood, and the tissue glucose value in a simple and at the same time less computationally intensive manner.

According to a further preferred embodiment of the invention, several earlier measured values are filtered, in particular by means of a “Kalman Fixed Interval Smoother”. A potential benefit of this is that historical readings are smoothed out, which improves the rate of change in blood glucose and therefore improves the accuracy of your current glucose reading.

According to a further preferred embodiment of the invention, when the “Kalman Fixed Interval Smoother” is used, it is run through forwards and backwards, with Kalman filtering taking place in the forward pass and an RTS filter and/or an MBF filter being used in the backward pass. This enables an efficient application of the “Kalman Fixed Interval Smoother”: The RTS filter or the “Extended RTS filter” in the case of a non-linear measurement model reduces the computation effort when using a stationary state transition model. The MBF filter reduces the computational effort for non-stationary state transition models.

According to a further preferred embodiment of the invention, the trend in blood sugar concentration is classified using a number of categories, in particular using at least seven categories. The trend or the future course of his blood sugar can thus be displayed to a user in a simple and efficient manner.

Further important features and advantages of the invention result from the dependent claims, from the drawings and from the associated description of the figures based on the drawings.

It goes without saying that the features mentioned above and those still to be explained below can be used not only in the combination specified in each case, but also in other combinations or on their own, without departing from the scope of the present invention.

Preferred designs and embodiments of the present invention are shown in the drawings and are explained in more detail in the following description. All transformation steps of equations, assumptions, solution methods, etc. can be used separately without departing from the scope of the invention.

### IN THE DRAWINGS

FIG. 1 shows in schematic form steps of a method according to an embodiment of the present invention; and

FIG. 2 shows a course of the blood sugar over time when using a Kalman filter and a Kalman smoother according to an embodiment of the present invention; and

FIG. 3 shows a course of the blood sugar over time when using a Kalman filter and a Kalman smoother according to an embodiment of the present invention with trend estimation.

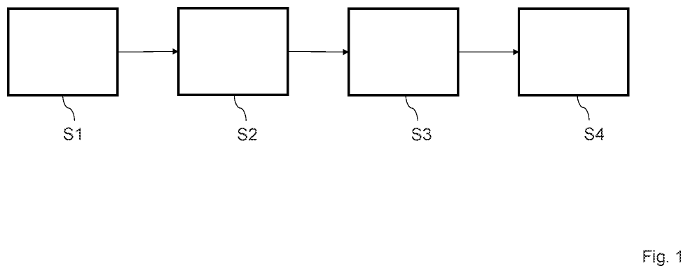

FIG. 1 shows in detail the steps of a method for, in particular, continuously determining a current glucose value in a transport fluid, in particular blood, of an organism to determine the glucose concentration in the blood based on the use of at least one Kalman filter in the case of a measurement model in the form of a linear function or using at least one extended Kalman filter in the case of a measurement model in the form of a nonlinear function.

The procedure comprises the following steps:

In a first step S1, a sensor device is used to determine a series of measurements, comprising at least two measured values spaced apart in time for a tissue glucose value in the tissue surrounding the transport fluid.

In a further step S2, the tissue glucose value is determined using the determined series of measurements based on a measurement model in the form of a linear or non-linear function, with the measurement model being used to assign measured values of the sensor device to tissue glucose values, taking into account at least one measurement noise value.

In a further step S3, at least one state transition model is provided, with the at least one state transition model being used to assign at least one glucose value in the transport fluid to the determined tissue glucose values, taking into account at least one process noise value.

In a further step S4, the current glucose value is estimated based on an approximation of at least one provided state transition model and the determined tissue glucose value using at least one Kalman filter in the case of a measurement model in the form of a linear function or at least one extended Kalman filter in the case of a measurement model in the form of a nonlinear function.

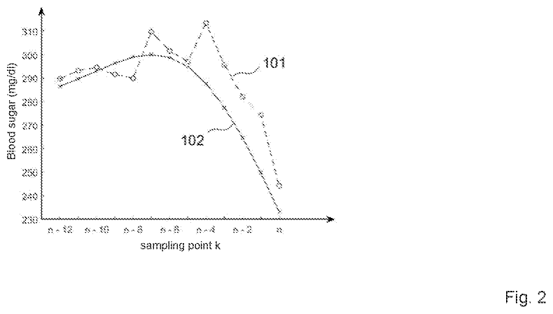

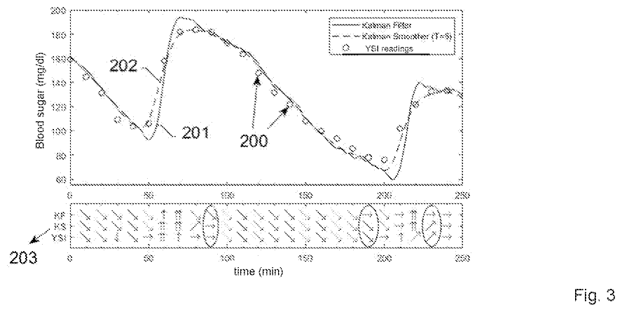

Further embodiments of the invention will now be explained in detail with reference to FIGS. 2 and 3, in particular also the use of different filters, the identification of outliners and the like. The respective features can be combined with one another in whole or in part.

The application of the Kalman filter is now described below:

The Kalman filter is an estimator for dynamic variables with Gaussian distributed measurement and process noise. Furthermore, the Markov-property that each state depends only on its previous state is required.

Any state vector

xₖ₊₁=Fₖxₖ+wₖ

is via the system matrix Fₖ, the previous state vector xₖ and the process noise wₖ defined.

The process noise generally has the covariance:

Qₖ=E{wₖwₖ^{T}}.

Further, it applies for the measurement vector

zₖ=Hₖxₖ+vₖ

with the measurement matrix Hₖ and the measurement noise \vec{v}ₖ. The following applies to the covariance of the measurement noise:

Rₖ=E{vₖvₖ^{T}}

The filter consists of two steps, a first prediction step and a second innovation step:

### 1) Prediction Step

\hat{x}_{k+1|k}=Fₖ\hat{x}_{k|k} State prediction

\hat{P}_{k+1|k}=Fₖ\hat{P}_{k|k}Fₖ^{T}+Qₖ Covariance prediction

\hat{z}_{k+1|k}=Hₖ₊₁\hat{x}_{k+1|k} Reading prediction

Sₖ₊₁=Hₖ₊₁\hat{P}_{k+1|k}Hₖ₊₁^{T}+Rₖ₊₁ Measurement covariance prediction

### 2) Innovation Step

Kₖ₊₁=\hat{P}_{k+1|k}+Hₖ₊₁^{T}Sₖ₊₁⁻¹ Filter reinforcement

\hat{x}_{k+1|k+1} =\hat{x}_{k+1|k}+Kₖ₊₁(zₖ₊₁−\hat{z}_{k+1|k}) State update

\hat{P}_{k+1|k+1}=\hat{P}_{k+1|k}−Kₖ₊₁Sₖ₊₁Kₖ₊₁^{T} Covariance update

The dynamics of tissue sugar—i.e. the change in the concentration of sugar in the tissue surrounding the blood vessel—is influenced by the diffusion of glucose from the blood. The modelling of the blood sugar dynamics is carried out in particular by modelling using uncertainties, since control variables such as food intake or insulin concentration are not known.

A simple possibility is to model the blood sugar assuming a constant blood sugar concentration

ġ^{b}=0+w.

Changes in the blood sugar concentration are then modelled using the process uncertainty w. Assuming that disturbances, such as an increase in the blood sugar concentration due to eating or a decrease due to insulin administration/release, have an influence on the blood sugar change, according to one embodiment of the present invention, changes in the blood sugar concentration are made using a model based on a constant blood sugar change

$${{g}^{b} ( \begin{matrix}{g}^{b} & \frac{{dg}^{b}}{dt}\end{matrix} )}^{T} and \\ {\dot{g}}^{b} = ( \begin{matrix}0 & 1 \\ 0 & 0\end{matrix} ) {g}^{b} + ( \begin{matrix}0 \\ W\end{matrix} ) .$$

Disturbances are modelled by a process error w in the blood sugar change.

For the discrete state space description with gₖ^{b}=(gₖ^{b}Δgₖ^{b})^{T} and the discrete blood sugar change Δgₖ^{b} it results

$${g}_{k + 1}^{b} = ( \begin{matrix}1 & Δ t \\ 0 & 1\end{matrix} ) {g}_{k}^{b} + ( \begin{matrix}0 \\ {W}_{k}\end{matrix} ) .$$

If the assumption that the process noise is uncorrelated, white noise is violated, the model can be expanded to meet to meet the requirements again. If the current process noise value correlates with historical process noise values, the Markov-property is violated.

With regard to the description of blood sugar dynamics, this is the case because blood sugar changes do not occur suddenly, but the decrease or increase in blood sugar becomes noticeable over a certain period of time. In a further embodiment of the present invention, this decaying behaviour can be represented by a model based on an exponential decay

$$\frac{{dg}^{b}}{dt} = \dot{{g}_{0}^{b}} {e}^{- α t}$$

valve induction with α<1 of the blood sugar change. The status transition matrix then results in

$$F = ( \begin{matrix}1 & ( 1 - {e}^{- αΔ t} ) / α \\ 0 & {e}^{- α Δ t}\end{matrix} )$$

and in approximation (series expansion

$${e}^{x} = {\sum }_{k = 0}^{∞} \frac{{x}^{k}}{k !}$$

up to k=1, e^{-αΔt}≈1−αΔt) to the pure damping with

$$F = ( \begin{matrix}1 & Δ t \\ 0 & a\end{matrix} ) .$$

In a further embodiment of the present invention, the dynamics of the blood sugar can be calculated using an autoregressive model (AR model) of order p

$${g}_{k}^{b} = c + {\sum }_{i = 1}^{p} {a}_{i} {g}_{k - i}^{b}$$

take place. The current value is modelled by the weighted sum of the previous values. It should be noted here that this modelling contradicts the requirement of the Kalman filter according to the Markov-property of the states that each state depends only on its previous one.

With a second-order model it follows with gₖ^{b}=(gₖ^{b}gₖ₋₁^{b})^{T} with c=0:

$${g}_{k + 1}^{b} = ( \begin{matrix}{a}_{1} & {a}_{2} \\ 0 & 1\end{matrix} ) {g}_{k}^{b} + ( \begin{matrix}{W}_{k} \\ 0\end{matrix} ) .$$

Choosing the parameters a₁=2 and a₂=−1, the AR model corresponds to a constant blood glucose change model with Δgₖ^{b}=gₖ^{b}−gₖ₋₁^{b}.

In order to model the diffusion process of the blood sugar in the blood into the surrounding tissue and then from the tissue into a sensor, in one embodiment of the present invention the diffusion process of the glucose from the blood into the tissue and then into the sensor is summarized by considering the processes as a series circuit

$$\frac{d {g}^{s}}{d t} = \frac{1}{τ} ( {g}^{b} - {g}^{s} )$$

Hereby g^{s} the concentration of glucose in the sensor and the time constant τ consists of the sum of the time constant of the diffusion of glucose from the blood into the tissue fluid and from the tissue fluid in the sensor.

The time-discrete formulation with sampling Δt results from applying the matrix exponential function to:

gₖ₊₁=(1−e^{-Δt/τ})gₖ^{b}+(Δt−τ(1−e^{—Δt/τ}))Δgₖ^{b}+e^{-Δt/τ}gₖ^{s}

By approximating the exponential function by the series expansion

$$( {e}^{x} = {\sum }_{k = 0}^{∞} \frac{{x}^{{κ}^{´}}}{k !}$$

to k=1) it results:

$${g}_{k + 1}^{s} = {g}_{k}^{i} + \frac{Δ t}{τ} ( {g}_{k}^{b} - {g}_{k}^{s} ) = \frac{Δ t}{τ} {g}_{k}^{b} + ( 1 - \frac{Δ t}{τ} ) {g}_{k}^{s}$$

The overall state vector

$${x}_{k} = ( \begin{matrix}{g}_{k}^{b} \\ {g}_{k}^{s}\end{matrix} )$$

consists of the blood sugar vector gₖ^{b} and the glucose value in the sensor g^{s} together.

| | Blood sugar | BG-IG | | |
|---|---|---|---|---|
| | dynamic | dynamic | F | Q |
| 1a | Constant change | accurate | $( \begin{matrix}1 & Δ t & 0 \\ 0 & 1 & 0 \\ 1 - {e}^{- Δ t / τ} & Δ t - τ ( 1 - {e}^{- Δ t / τ} ) & {e}^{- Δ t / τ}\end{matrix} )$ | $( \begin{matrix}0 & 0 & 0 \\ 0 & Δ t {σ}_{w} & 0 \\ 0 & 0 & 0\end{matrix} )$ |
| 1b | | approximation | $( \begin{matrix}1 & Δ t & 0 \\ 0 & 1 & 0 \\ Δ t / τ & 0 & 1 - Δ t / τ\end{matrix} )$ | $( \begin{matrix}0 & 0 & 0 \\ 0 & Δ t {σ}_{w} & 0 \\ 0 & 0 & 0\end{matrix} )$ |
| 2a | Exponential decrease | accurate | $( \begin{matrix}1 & ( 1 - {e}^{- α Δ t} ) / α & 0 \\ 0 & {e}^{- α Δ t} & 0 \\ 1 - {e}^{- Δ t / τ} & \frac{( {e}^{- α Δ t} + α τ ( 1 - {e}^{- Δ t / τ} ) )}{α ( α τ - 1 )} & {e}^{- Δ t / τ}\end{matrix} )$ | $( \begin{matrix}0 & 0 & 0 \\ 0 & Δ t {σ}_{w} & 0 \\ 0 & 0 & 0\end{matrix} )$ |
| 2b | | approximation | $( \begin{matrix}1 & ( 1 - {e}^{- α Δ t} ) / α & 0 \\ 0 & {e}^{- α Δ t} & 0 \\ Δ t / τ & 0 & 1 - Δ t / τ\end{matrix} )$ | $( \begin{matrix}0 & 0 & 0 \\ 0 & Δ t {σ}_{w} & 0 \\ 0 & 0 & 0\end{matrix} )$ |
| 3 | Damping | Approximation | $( \begin{matrix}1 & Δ t & 0 \\ 0 & α & 0 \\ Δ t / τ & 0 & 1 - Δ t / τ\end{matrix} )$ | $( \begin{matrix}0 & 0 & 0 \\ 0 & Δ t {σ}_{w} & 0 \\ 0 & 0 & 0\end{matrix} )$ |
| 4 | AR- process | Approximation | $( \begin{matrix}{a}_{1} & {a}_{2} & 0 \\ 0 & 1 & 0 \\ Δ t / τ & 0 & 1 - Δ t / τ\end{matrix} )$ | $( \begin{matrix}Δ t {σ}_{w} & 0 & 0 \\ 0 & 0 & 0 \\ 0 & 0 & 0\end{matrix} )$ |

To determine the tissue glucose value, measured values of the sensor are assigned to tissue glucose values using a series of measurements based on a measurement model in the form of a linear or non-linear function using the measurement model, taking into account at least one measurement noise value. Various measurement models—based on a linear function or a non-linear function—according to embodiments of the invention are available for this purpose.

For a linear measurement model, with the sensitivity e and the offset o

zₖ=(0 0 e)xₖ+o+vₖ

when using the nonlinear measurement model based on a nonlinear function with weak nonlinearity, for example

$$h ( {g}^{s} ) = \frac{a \cdot {g}^{s} + b \cdot c}{{g}^{s} + c} ,$$

instead or the Kalman filter, the extended Kalman Filter is used. For this purpose, the measurement matrix Hₖ can be approximated by a first-order Taylor polynomial

$${z}_{k} = ( \begin{matrix}0 & 0 & {e}_{k} ) {x}_{k} + {o}_{k} + {v}_{k}\end{matrix}$$

$$with$$

$${e}_{k} = \frac{dh}{{dg}^{s}} {\vert}_{{g}^{s} = {g}_{k \vert k - 1}^{s}}$$

$$and$$

$${o}_{k} = h ( {g}^{s} = {g}_{k \vert k - 1}^{s} ) .$$

One problem with estimating blood sugar from continuous measurement data, so-called continuous glucose monitoring data—CGM data for short—is that changes in blood sugar, for example due to food intake or the effect of insulin, only become noticeable in the tissue fluid after a time delay of more than 10 minutes. On the one hand, this is due to physiological reasons. On the other hand, additional time is required until the tissue fluid has then diffused into the sensor for measurement. As a result, when the blood sugar increases, the estimated value first lags behind and then, if there is also a change in the tissue sugar, it rises very steeply, which represents non-physiological behaviour.

For a certain period of time, depending on the food eaten and the administration of insulin, the blood sugar then rises almost constantly until the insulin takes effect. The change decreases from there until the blood sugar decreases at a nearly constant rate. For healthy people, this process can be easily predicted due to the body's control loop. For diabetic patients, this cannot be determined without knowing about the administration of insulin, i.e. in particular the point in time and insulin-specific parameters such as the effective time.

Another change in blood glucose change occurs when the insulin wears off. This point in time can also only be determined with additional knowledge about the amount of insulin, duration of action, etc. This can result in a dynamic model with a constant blood sugar change leading to a significant overestimation of the blood sugar when the blood sugar rises and a corresponding underestimation when the blood sugar decreases.

The effect can be reduced by changing, in particular in a controlled manner, between the dynamic models.

For example, in one embodiment of the present invention, it is possible to switch back and forth between a constant rate of change (cROC) model with modelling of the uncertainty due to process noise and a constant blood glucose model (cBG) depending on certain parameters:

$$BZ - Dynamic = \{ \begin{matrix}cBZ & ( ROC < 0 \& BZ < {BZ}_{u} ) \vert ( ROC > 0 \& BZ > {BZ}_{o} ) \\ cROC & else\end{matrix}$$

A cBZ dynamic model is selected in particular when a predefinable lower blood sugar value BZᵤ is not reached or if the blood sugar concentration rises (ROC>0) and an upper blood sugar limit BZₒ is exceeded.

In one embodiment of the present invention—in order to further improve the estimation of the blood sugar content—a particularly continuous adjustment of the measurement noise is undertaken, which is assumed to be free of mean values and Gaussian distributed. This makes it possible to take into account variances between different sensors and the aging of the sensors. An adjustment or an update of the variances leads directly to a change, in particular an improvement, in the quality of the estimation of the current blood sugar value. However, if the measurement noise is underestimated, this leads to a very noisy measurement signal and thus to erroneous measurement values. On the other hand, if the measurement noise is estimated too high or the process noise is estimated too low, this leads to a time-delayed estimation, which also reduces the accuracy of the determination of the current blood sugar value.

In one embodiment of the present invention, a lower limit value for the variance is first determined for this purpose. This corresponds to the minimum variance of the measuring system resulting from physical and technical considerations. An initial value for the measurement variance is set based on the expected measurement variance.

To determine the variance of the measurement noise, the noise from a series of measurements made up of N measured values and the associated filtered measured values is first calculated

vₖ=zₖ−\hat{z}ₖ

whereby the measured values are filtered by means of a Kalman smoother. A Kolmogorow-Smimow-test at the significance level α can then be used to test whether the sample is a mean-free sample

$$( \overline{v} = \frac{1}{N} {\sum }_{k = 1}^{N} {v}_{k} = 0 )$$

distribution with the measurement variance σₖ follows.

The variance becomes independent of the test result by the sample variance of the named sample

$${σ}_{v}^{′} = \frac{1}{N * df} {\sum }_{k = 1}^{N} {ν}_{k}^{2}$$

where df is the number of degrees of freedom in the filtering.

If the null hypothesis of the Kolmogorow-Smirnow-test is rejected at the significance level α, the noise of the (next) N measured values is determined and the variance is replaced by the sample variance of the sample until the null hypothesis is no longer rejected.

In another embodiment, outliners in the measurements are detected. The so-called “Normalized Innovation Squared Value”, NIS value for short (consistency estimation), can be used to detect outliners. This tests whether the “Normalized Innovation Squared” (NIS)

ϵ_{γ}(k)=γ(k)^{T}S(k)⁻¹γ(k)

with the innovation γ(k)=zₖ₊₁−\hat{z}_{k+1|k} of an χ²distribution with dim[z] freedom-straight follows. For this purpose, in particular a one-sided χ²-test at the significance level α

ϵ_{γ}<r(1−α, dim[z])

is performed (for dim[z]=1 and α=0.05 is r=7.88).

Model errors, for example a strong increase when a blood sugar increase also appears in the tissue signal, can also lead to the limit value being exceeded, so that measured values are incorrectly recognized as outliners by the NIS test. Subsequently, these values are filtered through the Kalman filter, although this is not mandatory.

In one embodiment of the present invention, the measurement signals can therefore also be checked in such a way that a measurement value is only identified as an outliner if it falls below and/or exceeds certain limit values. Such outliners in the measured values are based on erroneous measurements, for example caused by pressure fluctuations, which are noticeable through a strong change in temperature.

In one embodiment of the present invention, several consecutive incorrect measurements can also be determined.

To identify these, the following procedure can be used

```
if (i>1)
            i = 0;
            break;
else if (measuredvalues(k) < lower_limit | measuredvalues(k) > upper_limit)
      if ϵ_{γ}(k) < r(1 − α, dim[z]) \\ NIS Test
            i = 0;
            continue;
        else
            i ++;
            break;
```

This allows to neither unnecessarily sort out measured values due to poor model description, nor erroneously sort out measured values for which the limit value condition is not met but the consistency of the Kalman filter is guaranteed. This allows in particular a restrictive selection of the respective limit values. In other words, the number of measured values incorrectly classified as outliners, in particular due to model uncertainties, can be reduced.

In one embodiment of the present invention, the following method is used: If the NIS outliner test is negative, but at least two previous measurements were classified as measurement errors (i>1), it is assumed that the system is malfunctioning and is only considered to be over when the limit values are met.

In one embodiment of the present invention, in the event that the limit value condition(s) are met, but at least two previous measured values were classified as measurement errors, the current measured value is sorted out. This enables relaxation processes to be taken into account. However, the counter i according to the above method is set to 0 again.

In one embodiment of the present invention, the number of arithmetic operations is reduced by approximating the BG-IG dynamics. Since the covariance of the prediction is symmetrical, it can be sufficient to determine six of the nine entries, which means that additional arithmetic operations can be saved.

In one embodiment of the present invention, the computing effort can be reduced if the state transition matrix, the measurement matrix and the covariance matrix of the process noise and the measurement noise are constant over a period of time, since in this case the covariance matrix of the prediction is opposite converges. If the change in the middle matrix element is below a limit value, the algorithm is reduced to calculating the state and measured value prediction as well as the updated state. In this way, only the prediction step and the innovation step for the state have to be carried out per step.

An extended Kalman filter, here in the form of a so-called “Kalman Fixed Interval Smoother”, is used to filter past measured values.

In addition to the possible benefits in calculating the rate of change of the blood glucose ROC and improving the estimation result, filtering past values can also have the benefit that the data can be used for the graphical display. Depending on the measurement noise of the Kalman filter, the blood sugar signal is relatively noisy, which is non-physiological (curve 101 in FIG. 2). This can only be reduced by a more conservative setting of the parameters, i.e. the variance of the measurement noise. However, this creates an additional time delay. However, by using the Kalman smoother, a smooth blood sugar signal (curve 102 of FIG. 2) can be provided without additional time delay.

“Kalman Fixed Interval Smoother” estimate the states in a fixed, past interval of length T from the measurements of this interval. They are based on the solution of the “Bayesian Optimal Smoothing” equations and consist of two-pass filters, with the forward pass corresponding to the Kalman filter. For the calculation of the smoothed states in the interval of length T in the backward run of the two-pass filter, the a priori and a posteriori state estimates \hat{x}_{k+1|k},\hat{x}_{k|k} are used and covariances of the prediction \hat{P}_{k+1|k},\hat{P}_{k|k} are stored.

In one embodiment of the present invention, the so-called Rauch-Tung-Striebel (RTS) smoother filter can be used for this purpose.

The RTS filter is based on the following filter equations, which are calculated for all n−T<k<n

\hat{x}_{k|n}=\hat{x}_{k|k}+Cₖ(\hat{x}_{k+1|n}−\hat{x}_{k+1|k})

\hat{P}_{k|n}=\hat{P}_{k|k}+Cₖ(\hat{P}_{k+1|n}−\hat{P}_{k+1|k})Cₖ^{T}

with Cₖ=\hat{P}_{k|k}Fₖ^{T}\hat{P}_{k+1|k}⁻¹ and the a priori and posteriori state estimates \hat{x}_{k+1|k},\hat{x}_{k|k} and covariances of the prediction \hat{P}_{k+1|k},\hat{P}_{k|k} at time k. For the initial initialization k=n−1 applies \hat{P}_{k+1|n}=\hat{P}_{n|n} and \hat{x}_{k+1|n}=\hat{x}_{n|n}, which corresponds to the current estimate and covariance of the prediction.

For a constant state transition model and converged covariance of the prediction P_{∞} applies to the matrix Cₖ=C_{∞}=P_{∞}FP_{∞}⁻¹ and \hat{P}_{k|n}=P_{∞}. In this case, the computational effort is thus after a single calculation of Cₖ reduced to the calculation of \hat{x}_{k|n}.

In a further embodiment, the so-called Extended Rauch-Tung-Striebel (ERTS) smoother can be used for a non-linear measurement model.

In a further embodiment, the so-called Modified Bryson Frazier (MBF) smoother can be used. This also uses the results of the Kalman filter in the forward run for smoothing in the reverse run. In contrast to the RTS smoother, the inverse of the matrix \hat{P}_{k+1|k}⁻¹ is not calculated with the MBF smoother, which for non-stationary state transition matrices or when changing covariance matrices (i.e. \hat{P}_{k|k}≠P₂₈ ) leads to significantly less computational effort.

The necessary calculation steps for the MBF filter are

\hat{x}_{k|n}=\hat{x}_{k|k-1}−\hat{P}_{k|k-1}λₖ

\hat{P}_{k|n}=\hat{P}_{k|k-1}+\hat{P}_{k|k-1}\hat{Λ}ₖ\hat{P}_{k|k-1}

λₖ=Cₖ^{T}λₖ₊₁−Hₖ^{T}Sₖ⁻¹ϵₖ

\hat{Λ}ₖ=Cₖ^{T}\hat{Λ}ₖ₊₁Cₖ+Hₖ^{T}Sₖ⁻¹Hₖ

with Cₖ=Fₖ(I−KₖHₖ) and the a priori state estimates and covariances \hat{P}_{k|k-1} and \hat{x}_{k|k-1} as well as the innovation ϵₖ from the Kalman filter. The MBF-Smoother is initialized with \hat{Λ}ₖ=0and λₖ=0.

In particular, the MBF filter can be used with a measurement model in the form of a non-linear function. The measurement model is approximated using a first-order Taylor polynomial

zₖ=(0 0 eₖ)xₖ+oₖ+vₖ

with

$${e}_{k} = \frac{d h}{{dg}^{s}} {\vert}_{{g}^{s} = {g}_{k}^{s} \vert k - 1} and {o}_{k} = h ( {g}^{s} = {g}_{k \vert k - 1}^{s} ) .$$

Estimating the blood sugar as accurately as possible is relevant for the calibration as well as for the evaluation of the data by a doctor. The optimization of the blood sugar estimation by the Kalman smoother reduces in particular overshoots and undershoots of the blood sugar, which is relevant for the analysis of hyperglycemic and hypoglycemic states, since these would otherwise be significantly overestimated (see FIG. 3).

When calibrating the entire system using self-monitoring of blood glucose data, i.e. blood glucose self-measurement SMBG, an accurate estimation of the blood glucose is also relevant, since estimation errors lead to poor identification of the sensor parameters. For example, in the case of a one-point calibration with an SMBG value and a corresponding blood glucose estimate, the following would apply to identify the offset in the linear sensor model: SMBG= [symbol] +o′. An erroneous estimate [symbol] =g^{b}+ϵ would thus lead to an error of ϵ in determining the offset.

One of the possible advantages of using the Kalman smoother online, i.e. using it continuously, compared to known post-processing of the data, is the computational efficiency. The last T results of the Kalman filter are required for the Kalman smoother. In the case of post-processing using the Kalman smoother, these would have to be determined again or stored completely. Other well-known methods such as regularized unfolding are significantly more computationally expensive.

In the event of changes in the blood sugar level, for example due to food intake or insulin, i.e. at the times at which the dynamic model changes the state can only be roughly reproduced, larger estimation errors also occur as a result. Using this data to estimate the mean blood glucose change over the last mΔt=10 minutes

$${ROC}_{k} = \frac{1}{m} {\sum }_{j = 0}^{m - 1} \frac{{g}_{k - j}^{b} - {g}_{k - ( j + 1 )}^{b}}{Δ t} = \frac{{g}_{k}^{b} - {g}_{k - m}^{b}}{Δ t}$$

this can lead to unphysiological values and noisy results. Thanks to the improved estimate of past values using the Kalman smoother, a more accurate estimate of the current blood sugar change can be made. The blood glucose change ROC results from the difference between the current KF blood glucose estimate and the filtered KS blood glucose value m sampling steps back:

$${ROC}_{k} = \frac{-}{m \cdot Δ t}$$

The current blood sugar change is visualized for the user by arrow symbols. The division takes place, for example, in five or seven groups (see table and FIG. 3)

| Symbol | ROC in mg/dl/min | Index i |
|---|---|---|
| [symbol] | >3 | 3 |
| ↑ | [2, 3] | 2 |
| [symbol] | [0.5, 2] | 1 |
| → | [−0.5, 0.5] | 0 |
| [symbol] | [−2, −0.5] | −1 |
| ↓ | [−2, −3] | −2 |
| [symbol] | <−3 | −3 |

On the one hand, the measured values 200 recorded are shown in FIG. 3. A comparison of a blood glucose profile 201 using a Kalman filter and a blood glucose profile 202 using a Kalman smoother shows that the estimation of the rate of change ROC using the filtered signal allows an improved classification or an improved trend (see reference number 203 in FIG. 3): The upper row of the classification 203 is based on the blood sugar profile 201, the middle row of the classification 203 is based on the blood sugar profile 202 and the lower row on the measured values 201. The measured values 201 represent the venous blood sugar values. These are measured here using a YSI 2300 Stat Plus meter. The circled arrows in row 203 indicate the more accurate classification of the trend using the Kalman smoother versus the Kalman filter.

In summary, at least one of the embodiments of the invention provides at least one of the following advantages and/or features:
Compensation of the time delay by modelling the diffusion process and estimating the current glucose value based on an approximation of at least one provided state transition model and the determined tissue glucose value using at least one Kalman filter in the case of a measurement model in the form of a linear function or at least one extended Kalman filter in the case of a measurement model in the form of a non-linear function.
Robustness against outliners in the measurement signal.
Adaptation of slowly changing model parameters.
Efficient implementation ensures high accuracy with low computational effort.
Time and computationally efficient estimation of blood glucose.
Adaptation of model parameters.
Increasing the robustness of the estimation by introducing restrictions.
Flexibility with regard to the measurement model, for example both linear and non-linear sensor models can be used and, if necessary, switched between these as required.
Low computing effort to protect the limited battery life.
Improved system calibration based on SMGB measurements.

Although the present invention has been described on the basis of preferred exemplary embodiments, it is not limited to them, but can be modified in many different ways.

### LIST OF REFERENCE SIGNS

101 Blood Glucose Kalman filter
102 Blood Sugar Kalman Smoother
200 Readings
201 Course of blood glucose using a Kalman filter
202 Course of blood glucose using a Kalman smoother
203 Classification/trend
S1-S4 Method steps

1. A method for, in particular, continuously determining a current glucose value in a transport fluid, in particular blood, of an organism, comprising the steps of
a) Determining (S1) a measurement series using a sensor device, comprising at least two measured values for a tissue glucose value that are spaced apart in time in the tissue surrounding the transport fluid,
b) determining (S2) the tissue glucose value using the determined series of measurements based on a measurement model in the form of a linear or non-linear function, with the measurement model measuring values of the sensor device measuring tissue glucose values taking into account at least one measurement noise value are assigned,
c) providing (S3) at least one state transition model, with the at least one state transition model being used to assign at least one glucose value in the transport fluid to the determined tissue glucose values, taking into account at least one process noise value, and
d) estimating (S4) the current glucose value in the transport fluid based on an approximation of at least one provided state transition model and the determined tissue glucose value using at least one Kalman filter in the case of a measurement model in the form of a linear function or at least one extended Kalman filter Case of a measurement model in the form of a non-linear function.

2. The method according to claim 1, characterized in that several state transition models are provided, which are changed depending on the course over time of the estimated current glucose value, in particular its rate of change over time.

3. The method according to claim 1, characterized in that at least two state transition models are provided, one on a constant glucose concentration, one based on a constant change in glucose concentration and/or one based on a weighted sum of previous glucose concentrations.

4. The method according to claim 1, characterized in that measured values determined are filtered by means of at least one filter function, errors, in particular measurement errors, of the sensor device being suppressed by means of the at least one filter function.

5. The method according to claim 1, characterized in that the at least one measurement noise value is adjusted, in particular regularly.

6. The method as claimed in claim 5, characterized in that, in order to adapt the at least one measurement noise value, the variance of the measurement noise is determined using a random sample of measured values, in particular it is estimated.

7. The method according to claim 6, characterized in that a statistical test, in particular a Kolmogorov-Smirnov-test, is used to check whether the null hypothesis—the sample follows a mean-free Gaussian distribution with the determined variance of the measurement noise—is not rejected.

8. The method according to claim 7, characterized in that the variance of the measurement noise is determined for at least one further sample of measured values as long as the null hypothesis is rejected.

9. The method according to claim 4, characterized in that measured values are checked for outliners using the at least one filter function and measured values that were determined as outliners are discarded, in particular using an NIS test.

10. The method as claimed in claim 9, characterized in that the measured values are checked using the at least one filter function to determine whether they are above or below specified limit values before they are discarded.

11. The method according to claim 10, characterized in that a current measured value that was not determined as an outliner is nevertheless rejected as a measurement error if at least a predetermined number, in particular two chronologically consecutive earlier measured values, were previously rejected as measurement errors.

12. The method according to claim 1, characterized in that the state transition model comprises a diffusion model for time-dependent modelling of the diffusion process of glucose from the transport fluid into the surrounding tissue.

13. The method according to claim 1, characterized in that several earlier measured values are filtered, in particular by means of a Kalman Fixed Interval Smoother.

14. The method according to claim 13, characterized in that when the Kalman Fixed Interval Smoother is used, it is run through forwards and backwards, with Kalman filtering being used in the forward pass and an RTS filter being used in the backward pass and/or of an MBF filter.

15. The method as claimed in claim 1, characterized in that the trend in blood sugar concentration is classified using a number of categories, in particular using at least seven categories.

16. Device for in particular continuously determining a current glucose value in a transport fluid, in particular blood, of an organism, in particular for carrying out a method according to claim 1, comprising
a sensor device, in particular for measuring fluorescence in the transport fluid surrounding tissue by means of a probe, in particular a polymer-optical fiber probe, designed to determine a series of measurements, comprising at least two measured values spaced apart in time for a tissue glucose value in the tissue surrounding the transport fluid,
a provision device, designed to provide at least one state transition model, with the at least one state transition model being used to assign at least one glucose value in the transport fluid to the determined tissue glucose values, taking into account at least one process noise value, and to provide a measurement model in the form of a linear or non-linear function, whereby measured values of the sensor device are assigned to tissue glucose values by means of the measurement model, taking into account at least one measurement noise value, and
an evaluation device, designed to determine the tissue glucose value using the determined series of measurements based on the measurement model and to estimate the current glucose value in the transport fluid based on an approximation of at least one provided state transition model and the determined tissue glucose value using at least one Kalman filter in the case of a measurement model in form of a linear function or at least an extended Kalman filter in the case of a measurement model in the form of a non-linear function.

17. Evaluation device for, in particular, continuously determining a current glucose value in a transport fluid, in particular blood, of an organism, comprising
at least one interface for connecting a sensor device for providing a series of measurements, comprising at least two measured values at different times for a tissue glucose value in the tissue surrounding the transport fluid,
at least one memory for storing at least one state transition model, wherein the at least one state transition model is used to assign the tissue glucose values determined by the at least one state transition model to at least one glucose value in the transport fluid to, taking into account at least one process noise value and for storing a measurement model in the form of a linear or non-linear function, with the measurement model being used to assign measured values of the sensor device to tissue glucose values, taking into account at least one measurement noise value, and
a computing device, designed to determine the tissue glucose value using the determined series of measurements based on the stored measurement model and for estimating the current glucose value in the transport fluid based on an approximation of at least one provided state transition model and the determined tissue glucose value using at least one Kalman filter in the case of a measurement model in the form of a linear function or at least one extended Kalman filter in the case of a measurement model in the form of a non-linear function.

18. Non-transitory, computer-readable medium for storing instructions which, executed on a computer, cause a method for, in particular, continuous determination of a current glucose value in a transport fluid, in particular blood, of an organism to be carried out, preferably suitable for carrying out a method according to claim 1, comprising the steps of
a) Determining by means of a sensor device a series of measurements comprising at least two time-spaced measured values for a tissue glucose value in the tissue surrounding the transport fluid,
b) determining the tissue glucose value using the determined series of measurements based on a measurement model in the form of a linear or non-linear function, with the measurement model being used to assign measured values of the sensor device to tissue glucose values, taking into account at least one measurement noise value,
c) providing at least one state transition model, with the at least one state transition model using the determined tissue glucose at least one glucose value in the transport fluid is assigned to glucose values, taking into account at least one process noise value, and
d) estimating the current glucose value in the transport fluid based on an approximation of at least one provided state transition model and the determined tissue glucose value using at least one Kalman filter in the case of a measurement model in the form of a linear function or at least one extended Kalman filter in the case of a measurement model in the form of a non-linear function.
